(19) United States
(12) Patent Application Publication
(10) Pub. No.: US 2022/0117906 A1
(43) Pub. Date: Apr. 21, 2022

(54) METHOD FOR SUPPRESSING COLD FLOW OF ACRYLIC PATCH

(71) Applicant: Hisamitsu Pharmaceutical Co., Inc., Tosu-shi, Saga (JP)

(72) Inventors: Ryo Tanaka, Tsukuba-shi, lbaraki (JP); Kazuya Kominami, Tsukuba-shi, lbaraki (JP); Naoyuki Uchida, Tsukuba-shi, lbaraki (JP); Takito Shima, Tsukuba-shi, lbaraki (JP)

(73) Assignee: Hisamitsu Pharmaceutical Co., Inc., Tosu-shi, Saga (JP)

(21) Appl. No.: 17/431,303

(22) PCT Filed: Jan. 23, 2020
(86) PCT No.: PCT/JP2020/002297

§ 371 Date: Aug. 16, 2021

(30) Foreign Application Priority Data
Feb. 18, 2019 (JP) 2019-026619

(51) Int. Cl. A61K 9/70; A61K 47/32

(52) U.S. Cl. CPC A61K 9/70; A61K 47/32

(57) ABSTRACT

The problem addressed by the present invention is to provide a method for suppressing cold flow during packaging or application, regardless of the properties or concentration of a drug, in a patch containing a drug and an acrylic adhesive base in an adhesive layer. The present invention relates to a method for suppressing cold flow of a patch equipped with a support layer and an adhesive layer containing a drug and an acrylic adhesive base, wherein the method causes calcium silicate to be contained in the adhesive layer.

### TECHNICAL FIELD

The present invention relates to a method for suppressing cold flow of a patch containing a drug and an acrylic-based adhesive base in an adhesive layer.

### BACKGROUND TECHNOLOGY

Regarding patches containing a drug and an acrylic-based adhesive base, several embodiments have been studied in addition to those using only an acrylic-based adhesive base as the adhesive; for example, those further added with a silicone adhesive (Patent Document 1), those further added with a rubber-based polymer (Patent Document 2) and the like have been proposed.

Meanwhile, in patches, it is generally known that plasticization of the adhesive matrix can cause occurrence of distortion, deformation or dimensional change under storage conditions, that is, occurrence of cold flow (Patent Document 3). Furthermore, it is known that drug release and transdermal absorbability can be controlled by including calcium silicate in patches (Patent Document 4).

### CITATION LIST

### Patent Document

[Patent Document 1] JP A No. 2002-510600
[Patent Document 2] WO 2014/159573
[Patent Document 3] JP A No. 2016-504360
[Patent Document 4] JP A No. 4-108739

### SUMMARY OF INVENTION

### Problems to be Solved by Invention

The present inventors have found that, while investigating a high-performance patch by means of including a drug and an acrylic-based adhesive base, depending on the physical properties and concentration of the drug in the adhesive base, cold flow tends to easily occur in the adhesive base layer during storage in a packaging material or during application of the patch. Therefore, the object of the present invention is to provide a method for suppressing cold flow during packaging or application regardless of physical properties and concentration.

### Means for Solving the Problems

The present inventors have conducted extensive studies to solve such problems, and found that, in a patch containing a drug and an acrylic-based adhesive base, cold flow of the patch is suppressed by adding calcium silicate to the adhesive layer, and as a result of further studies, the present inventors have completed the present invention. That is, the present invention relates to the following.

[1] A method for suppressing cold flow of a patch comprising a backing layer, and an adhesive layer containing a drug and an acrylic-based adhesive base, wherein the calcium silicate is contained in the adhesive layer.
[2] The method according to [1], wherein calcium silicate is contained in a proportion of 0.05 to 15 mass % relative to the total amount of the adhesive layer.
[3] The method according to [1] or [2], wherein the drug is contained in a proportion of 10 to 35 mass % relative to the total amount of the adhesive layer.
[4] The method according to [3], wherein the drug has a low melting point or is a drug which needs to be contained at a high concentration in the adhesive base.
[5] The method according to any one of [1]-[4], further for suppressing stringing and/or plaster residue.

### Advantageous Effects of Invention

According to the present invention, it is possible to suppress cold flow during packaging or application. By this, it is possible to obtain a patch containing an acrylic-based adhesive base which is excellent in handling, and which can provide sufficient and sustained medicinal effect by maintaining stability of the drug in the patch during storage and maintaining an appropriate dosage form over a long period of time. In particular, even a patch containing methylphenidate that has, as a drug, a low melting point at high concentration in the adhesive layer, it is possible to obtain a methylphenidate-containing patch excellent in handleability as described above.

### EMBODIMENTS FOR CARRYING OUT INVENTION

The patch of the present invention comprises, for example, a backing layer and an adhesive layer laminated on the backing layer.

The backing may be any one that can maintain the shape of the patch, especially the adhesive layer. Examples of a material of the backing include polyethylene, polypropylene, polybutadiene, ethylene-vinyl chloride copolymer, polyvinyl chloride, polyamide such as Nylon (trade name), polyester, cellulose derivative, synthetic resin such as polyurethane, etc. Nature of the backing is, for example, a film, a sheet, a sheet-like porous body, a sheet-like foam, a textile such as a woven fabric, a knitted fabric and a non-woven fabric, etc., and a laminate thereof. The thickness of the backing is not particularly limited, and usually it is preferably about 2 to 3000 μm.

The adhesive layer contains a drug, an acrylic-based adhesive base and calcium silicate. Furthermore, the patch of the present invention may contain, in addition to the drug, acrylic-based adhesive base and calcium silicate, if necessary, a plasticizer, an absorption promoter, a stabilizer, a solubilizer, a crosslinking agent, a preservative, a filler, and other additive components such as fragrances.

The drug used in the present invention is not particularly limited. Examples thereof include: hypnotics/sedatives (flurazepam hydrochloride, rilmazafone hydrochloride, etc.), antipyretic and anti-inflammatory analgesics (butorphanol tartrate, perisoxal citrate, etc.), stimulants (methamphetamine hydrochloride, methylphenidate, etc.), psychotropic agents (chlorpromazine hydrochloride, imipramine hydrochloride, risperidone, olanzapine, etc.), local anesthetics (lidocaine hydrochloride, procaine hydrochloride, etc.), urinary organ agents (oxybutynin), skeletal muscle relaxants (tizanidine hydrochloride, eperisone hydrochloride, pridinol mesylate, etc.), agents for autonomic nerves (carpronium chloride, neostigmine bromide, etc.), anti-Parkinson's agents (trihexyphenidyl hydrochloride, amantadine hydrochloride, etc.), antihistamines (cremastine fumarate, diphenhydramine tannate, etc.), bronchodilators (tulobuterol hydrochloride, procaterol hydrochloride, etc.), cardiotonic agents (isoprenaline hydrochloride, dopamine hydrochloride, etc.), coronary vasodilators (diltiazem hydrochloride, verapamil hydrochloride, etc.), peripheral vasodilators (nicametate citrate, trazoline hydrochloride, etc.), cardiovascular agents (flunarizine hydrochloride, nicardipine hydrochloride, etc.), antiarrhythmic agents (propranolol hydrochloride, alprenolol hydrochloride, etc.), anti-allergic agents (ketotifen fumarate, azelastine hydrochloride, etc.), antivertigo agents (betahistine mesilate, diphenidol hydrochloride, etc.), serotonin receptor antagonist antiemetics, narcotic analgesics (morphine sulfate, fentanyl citrate, etc.), selective β1 blockers (bisoprolol), analgesic and anti-inflammatory agents (ketoprofen), angiotensin converting enzyme inhibitors (captopril), antihypertensive agents (clonidine), and the like; and methylphenidate is particularly preferable.

In the present invention, a drug that has a low melting point or that needs to be contained in a high concentration in the adhesive layer is preferable. The drug having a low melting point is a drug having a melting point of 150° C. or lower. The drug may be in a free form, or may be an addition salt with a pharmaceutically acceptable acid or base. In the present invention, the melting point of the drug may be 150° C. or lower, 120° C. or lower, and 100° C. or lower. The melting point of the drug is preferably 80° C. or lower, more preferably 50° C. or lower. Examples of drugs with a low melting point include bisoprolol, oxybutynin, captopril, clonidine, ethyl aminobenzoate, ebastine, epirizole, emorfazone, gabexate mesylate, quinineethyl carbonate, chloramphenicol palmitate, chlorphenesin carbamate, ketoprofen, cholecalciferol, dibucaine hydrochloride, tacalcitol hydrate, tropicamide, fludiazepam, perphenazine, pentoxyverine citrate, miconazole, ibudilast, ibuprofen, ethosuximide, guaifenesin, cyanamide, cyclophosphamide hydrate, disulfiram, testosterone enanthate, trimethadione, nabumetone, metyrapone, methenolone enanthate, menatetrenone, ubidecarenone, amyl nitrite, isoflurane, enflurane, methyl salicylate, diphenhydramine, sevoflurane, tocopherol nicotinate, tocopherol succinate, nitroglycerin, nicotine, isosorbide nitrate, scopolamine, rotigotine, rivastigmine and the like. In addition, that the high concentration of a drug is contained in the adhesive means that the concentration of the drug in the adhesive layer may be 10 mass % or more, 15 mass % or more, and 20 mass % or more, or may be 25 mass % or more.

The content of the drug can be appropriately set by those skilled in the art, and based on the total amount of the adhesive layer, the concentration of the drug in the adhesive layer is preferably 10 to 35 mass %, more preferably 15 to 30 mass %, furthermore preferably 18 to 27 mass %, and particularly preferably 20 to 25 mass %.

The methylphenidate may be any isomers of methylphenidate including stereoisomers (d-erythro-methylphenidate, 1-erythro-methylphenidate, d-threo-methylphenidate, and 1-threo-methylphenidate), or a derivative or salt thereof; in addition it may be interchangeable with methylphenyl(piperidin-2-yl)acetate, and may be a derivative or salt thereof. The methylphenidate of the present invention may also be a mixture of two or more racemic compounds (such as d/l-erythro-methylphenidate and d/l-threo-methylphenidate).

The content of the above methylphenidate can be appropriately set by those skilled in the art, and based on the total amount of the adhesive layer, it is preferably 10 to 35 mass %, more preferably 15 to 30 mass %, furthermore preferably 18 to 27 mass %, and particularly preferably 20 to 25 mass %.

The patch of the present invention contains calcium silicate. By containing calcium silicate, it is possible not only to suppress cold flow of the patch, but also to suppress stringing and plaster residue at the time of peeling, and to obtain excellent physical properties as a preparation and handleability. As the calcium silicate, for example, a porous one can also be used. Specifically, Florite® R (by Tomita Pharmaceutical Co., Ltd., trade name), SIPERANT® 880 (by Evonik Industries AG, trade name), Calcium Silicate (by Spectrum Chemical Co., Ltd., trade name) and the like can be used. The content of calcium silicate can be appropriately set by those skilled in the art in consideration of sufficient preparation characteristics of the patch, and based on the total amount of the adhesive layer, it may be 0.05 to 15 mass %, and it is preferably 0.1 to 15 mass %, more preferably 0.5 to 10 mass %, furthermore preferably 1 to 5 mass %, and particularly preferably 2.5 to 5 mass %.

The acrylic-based adhesive base of the present invention is a component that imparts adhesiveness to the adhesive layer, and is, for example, a (co)polymer of one or two or more (meth)acrylic acid alkyl esters. Examples of alkyl (meth)acrylic acid alkyl esters include butyl (meth)acrylate, isobutyl (meth)acrylate, hexyl (meth)acrylate, octyl (meth)acrylate, 2-ethylhexyl (meth)acrylate, decyl (meth)acrylate, etc. In the present specification, the term “(meth)acrylic acid” means either one or both of acrylic acid and methacrylic acid, and similar expressions are defined similarly.

The acrylic-based adhesive base may be a copolymer formed from a (meth)acrylic acid alkyl ester (main monomer) and a comonomer. Examples of the main monomer include methyl (meth)acrylate, ethyl (meth)acrylate, butyl (meth)acrylate, hexyl (meth)acrylate, heptyl (meth)acrylate, octyl (meth)acrylate, 2-ethylhexyl (meth)acrylate, etc., and one of these may be used alone or two or more thereof may be used in combination. The comonomer may be any component that can be copolymerized with a (meth)acrylic acid alkyl ester. Examples of the comonomer include (meth)acrylic acid hydroxyalkyl ester, ethylene, propylene, styrene, vinyl acetate, N-vinylpyrrolidone, (meth)acrylic acid, (meth)acrylic acid amide, etc. The comonomer may be a single kind or a combination of two or more kinds.

Specific examples of the acrylic-based adhesive base include acrylic acid/acrylic acid octyl ester copolymer, 2-ethylhexyl acrylate/vinylpyrrolidone copolymer solution, acrylic acid ester/vinyl acetate copolymer, 2-ethylhexyl acrylate/2-ethylhexyl methacrylate/dodecyl methacrylate copolymer, methyl acrylate/2-ethylhexyl acrylate copolymer resin emulsion, and an acrylic-based polymer contained in an acrylic resin alkanolamine solution, etc. Specific examples of such an acrylic-based adhesive include DURO-TAK series (by Henkel) such as DURO-TAK® 387-2510, DURO-TAK® 87-2510, DURO-TAK® 387-2287, and DURO-TAK® 87-2287, DURO-TAK® 87-4287, DURO-TAK® 387-2516, DURO-TAK® 87-2516, DURO-TAK® 87-2074, DURO-TAK® 87-900A, DURO-TAK® 87-901A, DURO-TAK® 87-9301, DURO-TAK® 87-4098, etc.; GELVA series (by Henkel) such as GELVA® GMS 788, GELVA® GMS 3083, GELVA® GMS 3253, etc.; MAS series (by CosMED Pharmaceutical Co., Ltd.) such as MAS 811 (trade name), MAS683 (trade name), etc.; Eudragit® series (by Evonik Industries AG), Nicasol® (by Nippon Carbide Industries Co., Inc.), Ultrasol® (by Aica Kogyo Co., Ltd.).

The above acrylic-based adhesive bases may be used alone or in combination of two or more. In addition, the content of the acrylic-based adhesive base can be appropriately set by those skilled in the art in consideration of the sufficient adhesion strength and local irritation at the time of peeling of the patch, and based on the total amount of the adhesive layer, it is preferably 50 to 90 mass %, more preferably 65 to 85 mass %, and particularly preferably 75 to 80 mass %.

The plasticizer may be any of those that impart flexibility to the adhesive layer. Examples of the plasticizer include mineral oils (for example, paraffin oil, naphthenic oil, aromatic oil), animal oils (for example, squalane, squalene), vegetable oils (for example, olive oil, camellia oil, castor oil, tall oil, peanut oil), silicone oils, dibasic acid esters (for example, dibutyl phthalate, dioctyl phthalate), liquid rubbers (for example, liquid polybutene, liquid polyisoprene), liquid fatty acid esters (for example, isopropyl myristate, hexyl laurate, diethyl sebacate, diisopropyl sebacate), polyhydric alcohols (for example, diethylene glycol, polyethylene glycol, propylene glycol, dipropylene glycol), triacetin, triethyl citrate, crotamiton and the like. The plasticizers may be used alone or in combination of two or more.

The above plasticizers may be used alone or in combination of two or more. In addition, the content of the plasticizer can be appropriately set by those skilled in the art in consideration of sufficient plasticity of the patch, and based on the total amount of the adhesive layer, it may be 0.2 to 35 mass %, 0.5 to 30 mass %, 1 to 25 mass %, and it is preferably 2 to 25 mass %.

The patch may further comprise a release liner. The release liner is laminated on the surface of the adhesive layer opposite to the backing. The provision of the release liner tends to reduce attachment of dust or the like to the adhesive layer during storage.

The material of the release liner is not particularly limited, and films generally known to those skilled in the art can be used. Examples of material for the release liner include polyesters such as polyethylene terephthalate and polyethylene naphthalate; polyolefins such as polyethylene and polypropylene; a film of polyvinyl chloride, polyvinylidene chloride, etc.; a laminated film of high-quality paper and polyolefins; a film of Nylon®, aluminum and the like. The material of the release liner is preferably polypropylene or polyethylene terephthalate.

Next, an example of the method for producing the patch of the present invention will be described.

First, a mixture for forming an adhesive layer is prepared. Using a mixer, the above-mentioned drug, the acrylic-based adhesive base, and other components are dissolved or dispersed in a solvent of the adhesive base to obtain a mixture for forming an adhesive layer.

As a solvent for the adhesive base, toluene, hexane, ethyl acetate, cyclohexane, heptane, butyl acetate, ethanol, methanol, xylene, isopropanol, etc. may be used. These may be appropriately selected depending on the components to be dissolved or dispersed, and one kind may be used alone, or two or more kinds may be mixed and used in combination.

Then, the obtained mixture for forming the adhesive layer is directly spread on a backing to form an adhesive layer, and then a release liner for protecting the adhesive layer is adhered to the adhesive layer; or said mixture is spread on a paper or film that has been subjected to a release treatment to form an adhesive layer, and then placing a backing on it and transfer the adhesive onto the backing by pressure bonding to obtain a patch.

### EXAMPLES

### <Cold Flow Test 1/Plaster Residue Test>

### Experimental Method

The patches containing the acrylic-based adhesive base shown in Table 1 (preparations containing plasticizer instead of drug) were prepared, and 10 cm² of each patch was applied to the thighs of 5 adult subjects; after 12 hours, the cold flow on the four sides of said preparation was visually observed. Furthermore, the patch was peeled off after the observation, and the plaster residue on the four sides at the patch application site of said preparation was visually observed and evaluated according to the following criteria.

### Criteria of Cold Flow Score:

0: No cold flow observed
1: Cold flow from approximately ⅛ of the entire perimeter
2: Cold flow from approximately 2/8 of the entire perimeter
3: Cold flow from approximately ⅜ of the entire perimeter
4: Cold flow from approximately 4/8 of the entire perimeter
5: Cold flow from approximately ⅝ of the entire perimeter
6: Cold flow from approximately 6/8 of the entire perimeter
7: Cold flow from approximately ⅞ of the entire perimeter
8: Cold flow from the entire perimeter

### Criteria of Plaster Residue Score:

0: No plaster residue observed
1: Plaster residue observed on approximately ⅛ of the entire perimeter
2: Plaster residue observed on approximately 2/8 of the entire perimeter
3: Plaster residue observed on approximately ⅜ of the entire perimeter
4: Plaster residue observed on approximately 4/8 of the entire perimeter
5: Plaster residue observed on approximately ⅝ of the entire perimeter
6: Plaster residue observed on approximately 6/8 of the entire perimeter
7: Plaster residue observed on approximately ⅞ of the entire perimeter
8: Plaster residue observed on the entire perimeter

### Experimental Result

Compared to the cold flow/plaster residue scores of the preparation without a filler, the cold flow/plaster residue scores of the preparations to which respective fillers were added were low as shown in Table 1; and in particular, those of the preparation to which 5% calcium silicate was added were the lowest, showing that the cold flow and plaster residue were best improved.

TABLE 1
Results of cold flow test 1/plaster residue test (preparations
containing plasticizer instead of drug)

| | | | | Cold | Plaster |
|---|---|---|---|---|---|
| | | | | flow | residue |
| | Base | Plasticizer | Filler | score | score |
| Comp. | 80% | 5% Liquid paraffin | None | 6.40 | 6.60 |
| Ex. 1 | MAS-811 | 15% Light liquid | | | |
| | | paraffin | | | |
| Comp. | 75% | 5% Liquid paraffin | 5% Hydrated | 5.20 | 5.00 |
| Ex. 2 | MAS-811 | 15% Light liquid | silicon | | |
| | | paraffin | dioxide | | |
| Comp. | 75% | 5% Liquid paraffin | 5% | 3.40 | 4.60 |
| Ex. 3 | MAS-811 | 15% Light liquid | Magnesium | | |
| | | paraffin | alumino- | | |
| | | | metasilicate | | |
| Ex. 1 | 75% | 5% Liquid paraffin | 5% Calcium | 2.00 | 2.60 |
| | MAS-811 | 15% Light liquid | silicate | | |
| | | paraffin | | | |

### <Cold Flow Test 2>

### Experimental Method

The patches shown in Table 2-1 were prepared, and they were punched out into a 2.5 cm² square, put in a packaging material and sealed, and then stored at 60° C. and a humidity of 75% for 1 day. After taking out, the packaging material was opened, and the cold flow of the preparation reaching to the inner surface of the packaging material was evaluated with n=3, and the degree of cold flow reaching to the packaging material was evaluated from the average value using the same criteria of the above-mentioned “criteria of cold flow score”.

### Experimental Result

Compared to the cold flow score of the preparation without a filler, the cold flow scores of the preparations to which respective fillers were added were the same or lower as shown in Table 2-1; and in particular, those of the preparations to which calcium silicate was added were the lowest, showing that the cold flow was best improved. Furthermore, comparison with the results of the cold flow test shown in Table 1 (preparations containing plasticizer instead of drug) indicated that even the methylphenidate-containing patches showed cold flow scores similar to the preparations containing a plasticizer instead of the drug, so such patches were found to improve the cold flow satisfactorily.

TABLE 2-1
Results of cold flow test 2 (patches containing methylphenidate)

| | | | | Cold |
|---|---|---|---|---|
| | | | | flow |
| | Base | Drug | Filler | score |
| Comp. | 80% | 20% Methylphenidate | None | 7.00 |
| Ex. 4 | MAS-811 | | | |
| Comp. | 77.5% | 20% Methylphenidate | 2.5% Hydrated | 7.00 |
| Ex. 5 | MAS-811 | | silicon dioxide | |
| Comp. | 75% | 20% Methylphenidate | 5% Hydrated | 6.00 |
| Ex. 6 | MAS-811 | | silicon dioxide | |
| Comp. | 77.5% | 20% Methylphenidate | 2.5% Magnesium | 5.66 |
| Ex. 7 | MAS-811 | | aluminometasilicate | |
| Comp. | 75% | 20% Methylphenidate | 5% Magnesium | 5.34 |
| Ex. 8 | MAS-811 | | aluminometasilicate | |
| Ex. 2 | 77.5% | 20% Methylphenidate | 2.5% Calcium | 3.66 |
| | MAS-811 | | silicate | |
| Ex. 3 | 75% | 20% Methylphenidate | 5% Calcium | 2.00 |
| | MAS-811 | | silicate | |

### <Cold Flow Test 3>

### Experimental Method

Patches were prepared using various drugs shown in Table 2-2, and evaluated in the same manner as the cold flow test 2.

### Experimental Result

As shown in Table 2-2, by containing calcium silicate, cold flow was improved satisfactorily, and a tendency of improvement depending on the content concentration was observed. It was clarified that this indicates a tendency depending on the content concentration without being influenced by the kind of drugs.

TABLE 2-2
Results of cold flow test 3 (patches containing various drugs)

| | | | | Cold |
|---|---|---|---|---|
| | | | | flow |
| | Base | Drug | Filler | score |
| Ex. 11 | 79.95% | 20% Methylphenidate | 0.05% Calcium | 4.00 |
| | MAS-811 | | silicate | |
| Ex. 12 | 79% | 20% Methylphenidate | 1% Calcium | 2.67 |
| | MAS-811 | | silicate | |
| Comp. | 90% | 10% Methylphenidate | None | 2.33 |
| Ex. 26 | MAS-811 | | | |
| Ex. 13 | 89% | 10% Methylphenidate | 1% Calcium | 0.75 |
| | MAS-811 | | silicate | |
| Ex. 14 | 87% | 10% Methylphenidate | 3% Calcium | 0.08 |
| | MAS-811 | | silicate | |
| Comp. | 90% | 10% Bisoprolol | None | 3.00 |
| Ex. 27 | MAS-811 | | | |
| Ex. 15 | 89% | 10% Bisoprolol | 1% Calcium | 1.67 |
| | MAS-811 | | silicate | |
| Ex. 16 | 87% | 10% Bisoprolol | 3% Calcium | 1.00 |
| | MAS-811 | | silicate | |
| Comp. | 90% | 10% Oxybutynin | None | 3.00 |
| Ex. 28 | MAS-811 | | | |
| Ex. 17 | 89% | 10% Oxybutynin | 1% Calcium | 1.50 |
| | MAS-811 | | silicate | |
| Ex. 18 | 87% | 10% Oxybutynin | 3% Calcium | 1.08 |
| | MAS-811 | | silicate | |
| Comp. | 90% | 10% Ketoprofen | None | 2.33 |
| Ex. 29 | MAS-811 | | | |
| Ex. 19 | 89% | 10% Ketoprofen | 1% Calcium | 0.75 |
| | MAS-811 | | silicate | |
| Ex. 20 | 87% | 10% Ketoprofen | 3% Calcium | 0.42 |
| | MAS-811 | | silicate | |
| Comp. | 90% | 10% Captopril | None | 1.00 |
| Ex. 30 | MAS-811 | | | |
| Ex. 21 | 89% | 10% Captopril | 1% Calcium | 0.08 |
| | MAS-811 | | silicate | |
| Ex. 22 | 87% | 10% Captopril | 3% Calcium | 0.08 |
| | MAS-811 | | silicate | |
| Comp. | 90% | 10% Clonidine | None | 1.00 |
| Ex. 31 | MAS-811 | | | |
| Ex. 23 | 89% | 10% Clonidine | 1% Calcium | 0.17 |
| | MAS-811 | | silicate | |
| Ex. 24 | 87% | 10% Clonidine | 3% Calcium | 0.00 |
| | MAS-811 | | silicate | |

### <Probe Tack Test 1>

### Experimental Method

A patch (backing=PET film, release liner=release-treated PET film) containing 20% methylphenidate, each filler and an acrylic-based adhesive base was prepared. In the probe tack test, the adhesive layer of the patch was brought into contact with a stainless steel probe (5 mmΦ) (speed=1.00 mm/sec, load=5 N/cm², time=1.00 sec), and then peeled (speed=1.00 mm/sec), and the following values of each patch were obtained to evaluate adhesive properties.

A: Time from the start of peeling to the end of peeling from the probe (sec)
B: Time from maximum load to the end of peeling (sec)
C: Maximum load (gf)

The test was carried out with n=3, and the values of A (sec), B (sec) and C (gf) were obtained for each evaluation sample, and C/B was calculated. The result of the calculation of each average value is shown in the table.

### Experimental Result

Acrylic-Based Adhesive Base MAS-811 Probe Tack Test (Methylphenidate)

As shown in Table 3-1, B time of the preparations added with each filler tended to decrease compared to that of the preparation without a filler. The B time varies depending on the degree of stringing of the adhesive base, but when comparison was made among the same kind of bases, those having a long B time exhibit stringing, indicating low cohesiveness. In particular, those containing calcium silicate had a shorter B time than other preparations containing Hydrated silicon dioxide or magnesium aluminometasilicate, and had excellent cohesiveness.

Furthermore, as shown in Table 3-1, the maximum load C/B time of the preparation with each filler tended to increase compared to the preparation without a filler. Maximum load C/B time shows a balance between cohesiveness and adhesiveness of the adhesive base, and when comparison is made for the same kind of bases, it indicates that one having a large maximum load C/B time shows good adhesiveness. In particular, those containing calcium silicate had a larger maximum load C/B time than those containing Hydrated silicon dioxide or magnesium aluminometasilicate, and their balance between cohesiveness and adhesiveness was excellent.

TABLE 3-1
Results of acrylic-based adhesive base MAS-811 probe tack test

| | Base | Drug | Filler | A(s) | B(s) | C(gf) | C/B |
|---|---|---|---|---|---|---|---|
| Comp. | 80% MAS-811 | 20% Methylphenidate | None | 1.315 | 0.574 | 483 | 907 |
| Ex. 4 | | | | | | | |
| Comp. | 77.5% MAS-811 | 20% Methylphenidate | 2.5% Hydrated silicon | 1.267 | 0.447 | 496 | 1112 |
| Ex. 5 | | | dioxide | | | | |
| Comp. | 75% MAS-811 | 20% Methylphenidate | 5% Hydrated silicon | 0.948 | 0.412 | 535 | 1309 |
| Ex. 6 | | | dioxide | | | | |
| Comp. | 77.5% MAS-811 | 20% Methylphenidate | 2.5% Magnesium | 1.182 | 0.448 | 607 | 1372 |
| Ex. 7 | | | aluminometasilicate | | | | |
| Comp. | 75% MAS-811 | 20% Methylphenidate | 5% Magnesium | 1.083 | 0.342 | 557 | 1704 |
| Ex. 8 | | | aluminometasilicate | | | | |
| Ex. 2 | 77.5% MAS-811 | 20% Methylphenidate | 2.5% Calcium silicate | 0.820 | 0.170 | 371 | 2180 |
| Ex. 3 | 75% MAS-811 | 20% Methylphenidate | 5% Calcium silicate | 0.838 | 0.173 | 348 | 2048 |

Acrylic-Based Adhesive Base MAS-811 Probe Tack Test (Various Drugs)

As shown in Table 3-2, when various drugs were used, with regard to methylphenidate, oxybutynin and ketoprofen, the preparations containing calcium silicate had a shorter B time than the preparations not containing calcium silicate, showing excellent cohesiveness. Furthermore, compared to the preparations not containing calcium silicate, the preparations containing calcium silicate had a larger maximum load C/B time and they were excellent in the balance between cohesiveness and adhesiveness.

TABLE 3-2
Results of acrylic-based adhesive base MAS-811 probe tack test

| | Base | Drug | Filler | A(s) | B(s) | C(gf) | C/B |
|---|---|---|---|---|---|---|---|
| Ex. 11 | 79.95% MAS-811 | 20% Methylphenidate | 0.05% Calcium silicate | 1.042 | 0.198 | 344.9 | 1750.9 |
| Ex. 12 | 79% MAS-811 | 20% Methylphenidate | 1% Calcium silicate | 0.953 | 0.163 | 304.0 | 1875.9 |
| Comp. | 90% MAS-811 | 10% Methylphenidate | None | 0.873 | 0.095 | 334.7 | 3547.8 |
| Ex. 26 | | | | | | | |
| Ex. 13 | 89% MAS-811 | 10% Methylphenidate | 1% Calcium silicate | 0.893 | 0.087 | 322.8 | 3816.2 |
| Ex. 14 | 87% MAS-811 | 10% Methylphenidate | 3% Calcium silicate | 0.692 | 0.057 | 228.6 | 4077.1 |
| Comp. | 90% MAS-811 | 10% Bisoprolol | None | 0.758 | 0.088 | 258.0 | 2923.7 |
| Ex. 27 | | | | | | | |
| Ex. 15 | 89% MAS-811 | 10% Bisoprolol | 1% Calcium silicate | 0.718 | 0.160 | 226.4 | 1753.9 |
| Ex. 16 | 87% MAS-811 | 10% Bisoprolol | 3% Calcium silicate | 0.685 | 0.078 | 175.7 | 2279.7 |
| Comp. | 90% MAS-811 | 10% Oxybutynin | None | 0.927 | 0.110 | 388.2 | 3605.3 |
| Ex. 28 | | | | | | | |
| Ex. 17 | 89% MAS-811 | 10% Oxybutynin | 1% Calcium silicate | 0.913 | 0.092 | 338.1 | 3720.5 |
| Ex. 18 | 87% MAS-811 | 10% Oxybutynin | 3% Calcium silicate | 0.862 | 0.083 | 298.4 | 3818.8 |
| Comp. | 90% MAS-811 | 10% Ketoprofen | None | 1.012 | 0.123 | 436.8 | 3577.1 |
| Ex. 29 | | | | | | | |
| Ex. 19 | 89% MAS-811 | 10% Ketoprofen | 1% Calcium silicate | 0.970 | 0.117 | 419.9 | 3609.5 |
| Ex. 20 | 87% MAS-811 | 10% Ketoprofen | 3% Calcium silicate | 0.792 | 0.057 | 291.2 | 5150.5 |
| Comp. | 90% MAS-811 | 10% Captopril | None | 0.907 | 0.050 | 343.3 | 7808.0 |
| Ex. 30 | | | | | | | |
| Ex. 21 | 89% MAS-811 | 10% Captopril | 1% Calcium silicate | 0.750 | 0.033 | 233.4 | 7010.3 |
| Ex. 22 | 87% MAS-811 | 10% Captopril | 3% Calcium silicate | 0.713 | 0.027 | 248.8 | 9450.7 |
| Comp. | 90% MAS-811 | 10% Clonidine | None | 0.797 | 0.052 | 370.5 | 9443.1 |
| Ex. 31 | | | | | | | |
| Ex. 23 | 89% MAS-811 | 10% Clonidine | 1% Calcium silicate | 0.668 | 0.052 | 258.9 | 5217.7 |
| Ex. 24 | 87% MAS-811 | 10% Clonidine | 3% Calcium silicate | 0.732 | 0.045 | 272.1 | 6181.5 |

Acrylic-Based Adhesive Base Duro-Tak87-900A Probe Tack Test

Regarding the other acrylic-based adhesives, the results were similar to those of MAS-811, as shown in Tables 4 to 6.

TABLE 4
Results of acrylic-based adhesive base Duro-Tak87-900A probe tack test

| | Base | Drug | Filler | A(s) | B(s) | C(gf) | C/B |
|---|---|---|---|---|---|---|---|
| Comp. | 80% Duro-Tak87-900A | 20% Methylphenidate | None | 4.418 | 3.588 | 534 | 246 |
| Ex. 9 | | | | | | | |
| Comp. | 77.5% Duro-Tak87-900A | 20% Methylphenidate | 2.5% Hydrated silicon | 2.210 | 1.532 | 507 | 329 |
| Ex. 10 | | | dioxide | | | | |
| Comp. | 75% Duro-Tak87-900A | 20% Methylphenidate | 5% Hydrated silicon | 3.980 | 3.220 | 569 | 224 |
| Ex. 11 | | | dioxide | | | | |
| Comp. | 77.5% Duro-Tak87-900A | 20% Methylphenidate | 2.5% Magnesium | 2.162 | 1.395 | 444 | 342 |
| Ex. 12 | | | aluminometasilicate | | | | |
| Comp. | 75% Duro-Tak87-900A | 20% Methylphenidate | 5% Magnesium | 1.747 | 1.130 | 578 | 588 |
| Ex. 13 | | | aluminometasilicate | | | | |
| Ex. 4 | 77.5% Duro-Tak87-900A | 20% Methylphenidate | 2.5% Calcium silicate | 0.618 | 0.245 | 339 | 1434 |
| Ex. 5 | 75% Duro-Tak87-900A | 20% Methylphenidate | 5% Calcium silicate | 0.665 | 0.182 | 404 | 2181 |

Acrylic-based adhesive base Duro-Tak87-4287 probe tack test

TABLE 5
Results of acrylic-based adhesive base Duro-Tak87-4287 probe tack test

| | Base | Drug | Filler | A(s) | B(s) | C(gf) | C/B |
|---|---|---|---|---|---|---|---|
| Comp. | 80% Duro-Tak87-4287 | 20% Methylphenidate | None | 3.457 | 2.840 | 458 | 164 |
| Ex. 14 | | | | | | | |
| Comp. | 77.5% Duro-Tak87-4287 | 20% Methylphenidate | 2.5% Hydrated silicon | 3.920 | 3.333 | 477 | 149 |
| Ex. 15 | | | dioxide | | | | |
| Comp. | 75% Duro-Tak87-4287 | 20% Methylphenidate | 5% Hydrated silicon | 2.875 | 2.238 | 476 | 215 |
| Ex. 16 | | | dioxide | | | | |
| Comp. | 77.5% Duro-Tak87-4287 | 20% Methylphenidate | 2.5% Magnesium | 4.597 | 3.918 | 477 | 127 |
| Ex. 17 | | | aluminometasilicate | | | | |
| Comp. | 75% Duro-Tak87-4287 | 20% Methylphenidate | 5% Magnesium | 2.323 | 1.742 | 542 | 327 |
| Ex. 18 | | | aluminometasilicate | | | | |
| Ex. 6 | 77.5% Duro-Tak87-4287 | 20% Methylphenidate | 2.5% Calcium silicate | 1.743 | 1.138 | 527 | 474 |
| Ex. 7 | 75% Duro-Tak87-4287 | 20% Methylphenidate | 5% Calcium silicate | 0.862 | 0.228 | 550 | 2560 |

Acrylic-Based Adhesive Base Duro-Tak87-2516 Probe Tack Test

TABLE 6
Results of acrylic-based adhesive base Duro-Tak87-2516 probe tack test

| | Base | Drug | Filler | A(s) | B(s) | C(gf) | C/B |
|---|---|---|---|---|---|---|---|
| Comp. | 80% Duro-Tak87-2516 | 20% Methylphenidate | None | 1.608 | 0.992 | 573 | 582 |
| Ex. 19 | | | | | | | |
| Comp. | 77.5% Duro-Tak87-2516 | 20% Methylphenidate | 2.5% Hydrated silicon | 1.685 | 1.122 | 509 | 457 |
| Ex. 20 | | | dioxide | | | | |
| Comp. | 75% Duro-Tak87-2516 | 20% Methylphenidate | 5% Hydrated silicon | 1.745 | 1.040 | 524 | 518 |
| Ex. 21 | | | dioxide | | | | |
| Comp. | 77.5% Duro-Tak87-2516 | 20% Methylphenidate | 2.5% Magnesium | 1.435 | 0.850 | 517 | 614 |
| Ex. 22 | | | aluminometasilicate | | | | |
| Comp. | 75% Duro-Tak87-2516 | 20% Methylphenidate | 5% Magnesium | 1.475 | 0.850 | 536 | 636 |
| Ex. 23 | | | aluminometasilicate | | | | |
| Ex. 8 | 77.5% Duro-Tak87-2516 | 20% Methylphenidate | 2.5% Calcium silicate | 1.020 | 0.422 | 589 | 1434 |
| Ex. 9 | 75% Duro-Tak87-2516 | 20% Methylphenidate | 5% Calcium silicate | 0.823 | 0.213 | 437 | 2087 |

### <Probe Tack Test 2>

### Experimental Method

The patches containing the acrylic-based adhesive base shown in Table 7 (preparations containing plasticizer instead of drug) were prepared, and a probe tack test was conducted in the same manner as the probe tack test 1.

### Experimental Result

Acrylic-Based Adhesive Base MAS-811 Probe Tack Test

As shown in Table 7, B time of the preparations added with each filler tended to decrease compared to those of the preparations without a filler. The B time varies depending on the degree of stringing of the adhesive base, but when comparison was made among the same kind of bases, those having a long B time exhibit stringing, indicating low cohesiveness. In particular, those containing calcium silicate had a shorter B time than other preparations containing Hydrated silicon dioxide or magnesium aluminometasilicate, and had excellent cohesiveness.

Furthermore, as shown in Table 7, the maximum load C/B time of the preparation with each filler tended to increase compared to the preparations without a filler. Maximum load C/B time shows a balance between cohesiveness and adhesiveness of the adhesive base, and when comparison is made for the same kind of bases, it indicates that one having a large maximum load C/B time shows good adhesiveness. In particular, those containing calcium silicate had a larger maximum load C/B time than those containing Hydrated silicon dioxide or magnesium aluminometasilicate, and their balance between cohesiveness and adhesiveness was excellent.

TABLE 7
Results of acrylic-based adhesive base MAS-811 probe tack test

| | Base | Drug/Plasticizer | Filler | A(s) | B(s) | C(gf) | C/B |
|---|---|---|---|---|---|---|---|
| Comp. | 80% MAS-811 | 20% Methylphenidate | None | 1.315 | 0.574 | 483 | 907 |
| Ex. 4 | | | | | | | |
| Comp. | 80% MAS-811 | 5% Liquid paraffin | None | 1.237 | 0.475 | 410 | 865 |
| Ex. 1 | | 15% Light liquid paraffin | | | | | |
| Comp. | 79% MAS-811 | 5% Liquid paraffin | 1% Hydrated silicon | 1.108 | 0.365 | 375 | 1029 |
| Ex. 24 | | 15% Light liquid paraffin | dioxide | | | | |
| Comp. | 75% MAS-811 | 5% Liquid paraffin | 5% Hydrated silicon | 0.982 | 0.247 | 363 | 1475 |
| Ex. 2 | | 15% Light liquid paraffin | dioxide | | | | |
| Comp. | 79% MAS-811 | 5% Liquid paraffin | 1% Magnesium | 1.337 | 0.572 | 409 | 717 |
| Ex. 25 | | 15% Light liquid paraffin | aluminometasilicate | | | | |
| Comp. | 75% MAS-811 | 5% Liquid paraffin | 5% Magnesium | 1.068 | 0.318 | 374 | 1175 |
| Ex. 3 | | 15% Light liquid paraffin | aluminometasilicate | | | | |
| Ex. 10 | 79% MAS-811 | 5% Liquid paraffin | 1% Calcium silicate | 1.012 | 0.260 | 385 | 1516 |
| | | 15% Light liquid paraffin | | | | | |
| Ex. 1 | 75% MAS-811 | 5% Liquid paraffin | 5% Calcium silicate | 0.833 | 0.132 | 353 | 2687 |
| | | 15% Light liquid paraffin | | | | | |

As described above, the patches of the present invention containing methylphenidate, oxybutynin, or ketoprofen showed excellent results in all of the cold flow test, the plaster residue test, and the probe tack test. Even when methyl salicylate, nitroglycerin, nicotine, isosorbide nitrate, rotigotine, or rivastigmine was used in place of the methylphenidate of Example 3, the patches of the present invention showed the same excellent effects in each of the above tests.

1. A method for suppressing cold flow of a patch comprising a backing layer, and an adhesive layer containing a drug and an acrylic-based adhesive base, wherein the calcium silicate is contained in the adhesive layer.

2. The method according to claim 1, wherein calcium silicate is contained in a proportion of 0.05 to 15 mass % relative to the total amount of the adhesive layer.

3. The method according to claim 1, wherein the drug is contained in a proportion of 10 to 35 mass % relative to the total amount of the adhesive layer.

4. The method according to claim 3, wherein the drug has a low melting point or is a drug which needs to be contained at a high concentration in the adhesive base.

5. The method according to claim 1, further for suppressing stringing and/or plaster residue.
